(19) United States
(12) Patent Application Publication
(10) Pub. No.: US 2016/0228341 A1
(43) Pub. Date: Aug. 11, 2016

(54) Dentifrice Composition Comprising Sintered Hydroxyapatite

(71) Applicant: GLAXO GROUP LIMITED, Brent ford Middlesex (GB)

(72) Inventors: Robert Anthony LUCAS, Wey bridge, Surrey (GB); Anthony John SMITH, Kinver (GB); Changxiang WANG, Birmingham (GB)

(21) Appl. No.: 15/022,977

(22) PCT Filed: Sep. 24, 2014
(86) PCT No.: PCT/EP2014/070289

§ 371 Date: Mar. 18, 2016

(30) Foreign Application Priority Data
Sep. 26, 2013 (GB) 1317105.3

(51) Int. Cl. A61K 8/24; A61K 8/21; A61K 8/60; A61K 8/86; A61K 8/19; A61K 8/42; A61K 8/46; A61K 8/73; A61K 8/29; A61Q 11/00; A61K 8/25

(52) U.S. Cl. CPC A61K 8/24; A61Q 11/00; A61K 8/21; A61K 8/60; A61K 8/86; A61K 8/25; A61K 8/42; A61K 8/463; A61K 8/73; A61K 8/29; A61K 8/19; A61K 2800/412; A61K 2800/592; A61K 2800/5922; A61K 2800/80; A61K 2800/28

(57) ABSTRACT

A dentifrice composition comprising a sintered hydroxyapatite abrasive agent providing good cleaning and whitening of the tooth surface, with minimal dentine abrasivity.

This invention relates to dentifrice compositions comprising a sintered hydroxyapatite as an abrasive agent which can effectively clean and whiten the enamel of teeth and the surface of dental prostheses without a high degree of abrasion and scratching of the tooth surface. Such dentifrice compositions thereby provide good cleaning of tooth surfaces.

Dentifrices are used to clean the teeth, and are generally in the form of a toothpaste used in conjunction with a toothbrush, on a daily basis. The toothpaste will aid in the removal of food particles and tooth discolouration caused by substances such as tobacco, tea or wine in addition to the removal of plaque from the surface of the teeth. Toothpastes may also polish the teeth. Cleaning and polishing the tooth surfaces are affected by (1) mechanical means such as abrasive substances and (2) chemical processes such as materials that modify the stained plaque.

Over-the-counter teeth whitening preparations have been developed to address the cosmetic preference of many to restore luster to tooth enamel discolored by surface entrapped materials; the term lightening may also be used in conjunction with the advertising and sale of these products. While all dentifrices and mouthwashes contain some cleaning and polishing agents some enamel deposits are not removed completely by these agents under normal use conditions. These preparations may not be formulated with the amount or type of agent required to remove the amount of stains and discoloration which build up due to excessive exposure to the staining agent. For example, smokers often develop discolored enamel because the tars and particulate in exhaled cigarette smoke collect on the teeth. And a number of comestibles can stain or discolor tooth enamel, tea being one example of a beverage where the tannins in the tea quickly deposit on the tooth enamel. Some medicinal agents may cause staining or discoloration via entrapment, though this is not a usual common cause of this type of staining.

Oral healthcare compositions containing water soluble polyphosphate (also known as condensed phosphate) salts such as tripolyphosphate salts, are known for use as chemical agents to clean and whiten the teeth.

WO95/17158 (SmithKline Beecham Corp) discloses and claims a composition for reducing or removing surface deposited stains from natural teeth and dental prostheses comprising a dentally acceptable preparation comprising about 5 to 15% by weight of a water soluble alkali metal tripolyphosphate. Suitably the water soluble alkali metal tripolyphosphate is sodium tripolyphosphate.

WO2005/027858 (Glaxo Group Ltd) relates to dentifrice compositions, in particular compositions comprising a fluoride source and a soluble calcium sequestering agent that is not an oxidising agent, for cleaning natural teeth and dentures. Such compositions show excellent cleaning properties whilst at the same time low abrasion characteristics. These compositions must have a Relative Dentine Abrasivity (RDA) value of below 30 and an in-vitro stain removal (IVSR) value greater than 50 (when compared to a control). The calcium sequestering agent, which is present in a proportion of 1-20 wt %, may be a condensed phosphate salt, such as sodium tripolyphosphate. An abrasive material may also be present in a proportion of 0-5 wt % of the composition.

U.S. Pat. No. 6,517,815 (Henkel Kommanditgesellschaft auf Aktien) discloses a dentifrice in the form of an aqueous paste or liquid dispersion, comprising 10% to 30% by weight of a combination of silica polishing agents and aluminium oxide in a ratio by weight of 10:0.2 to 10:2, 20% to 50% by weight of a humectants and 2% to 12% by weight of a condensed phosphate. The condensed phosphates are in the form of an alkali metal or ammonium salt. The aluminium oxide is preferably a lightly calcined alumina with a content of at least 10% by weight of α-aluminium oxide of various γ-aluminium oxides. It is suggested that the special combination of polishing agents (ie the combination of the silica and the alumina) is able to provide a dentifrice having good polishing and cleaning effects with only moderate dentine and enamel abrasion, notwithstanding the presence of the hard alumina polishing component.

U.S. Pat. No. 4,632,826 (Henkel Kommanditgesellschaft auf Aktien) discloses a tooth cream comprising 100 parts by weight of silica polishing agent and 2 to 15 parts by weight of weakly calcined alumina polishing agent. The silica polishing agent consists essentially of silica hydrogel and precipitated silica and the weakly calcined alumina 10 to 50% by weight of gamma aluminium oxide and 50 to 90% by weight of alpha aluminium oxide. The tooth cream is suitable for removing stain, polishing and cleaning the surface of teeth without producing any deep scratches or damage by daily use.

Klüppel et al. J. Soc. Cosmet. Chem., 37, 211-223 (July/August 1986) “Parameters for assessing the cleaning power of toothpastes” compares a number of dentifrice formulations for polishing and scratching effects. Test formulations are described containing as the sole abrasive material either a polishing alumina or a hydrated silica or a mixture of a hydrated silica with a polishing alumina. The results suggest that dentifrice products can be developed with high cleaning power and low dentine abrasion. Whilst the test formulations with increasing amounts of a polishing alumina, as the sole abrasive, can provide good cleaning power this was coupled with an unacceptable increase in enamel abrasion. The best test formulation contained a mixture of a silica abrasive and an alumina abrasive which exhibited a high cleaning value together with an unexpectedly low enamel abrasion value.

EP 0 029 332 A1 (Dental Chemical Co) discloses dentifrice compositions containing synthetic hydroxyapatite powder, described as being very effective in eliminating plaque from teeth and particularly when containing 0.1 to 20% by weight of NaCl and/or KCl and 0.003 to 3% by weight of MgCl₂, have a fortifying and remineralizing effect on tooth surfaces.

U.S. Pat. No. 4,933,171 (Bristow et al) describes compositions comprising an agent for desensitizing sensitive teeth such as potassium nitrate or strontium acetate, and a particulate abrasive material which is hydroxyapatite.

U.S. Pat. No. 6,919,070 B1 (Rudin et al) describes a stomatic composition having particles of hydroxyapatite with an average particle size in length (1), width (d) and thickness (h) of: 1 from 0.2 μm to about 0.0 1μm, d from about from 0.1 μm to about 0.001 μm and h from about from 0.1 μm to about 0.001 μm. Compositions therein are described as being of use for preventive treatment of caries, parodenitis and paradentosis.

It remains highly desirable to provide dentifrices that restore teeth to their natural white colour, dissolving and lifting away stains without abrasive scrubbing or bleaching.

It has now been found that sintered hydroxyapatite exhibits highly effective cleaning power, whilst at the same time providing low dentine abrasion to the tooth surface.

Accordingly in a first aspect the present invention provides a dentifrice composition comprising a sintered hydroxyapatite abrasive agent. A dentifrice composition according to the present invention will further comprise an orally acceptable carrier or excipient.

In an alternative aspect there is provided the use of a sintered hydroxyapatite as an abrasive agent in a dentifrice composition.

Hydroxyapatite (HA) is a member of the apatite group of minerals and has the chemical formula Ca₁₀(PO₄)₆(OH)₂. It is essentially a calcium phosphate including hydroxide having a Ca:P ratio of about 1.67:1. The hydoxyapatite of use in the present invention is sintered hydroxyapatite i.e. hydroxyapatite that has undergone a sintering process. Suitably the hydroxyapatite is sintered at a temperature of about 1250° C.

Suitably sintered hydroxyapatite of use in the invention is anhydrous or essentially anhydrous i.e. is free or substantially free of any bound and/or unbound water, although low amounts of water can be tolerated, for example up to about 1.5% by weight or less, e.g. in the range 0.001-1.5% by weight of the hydroxyapatite. This is in contrast to non-sintered hydroxyapatite preparations which have a higher percentage amount of water, for example from about 2.5% to about 5%, depending upon the drying temperature.

Suitably the sintered hydroxyapatite comprises a low specific BET surface area, as determined by the BET method using nitrogen gas as the adsorbant, for example less than about 20m²/g. Suitably the sintered hydroxyapatite exhibits a minimum hardness of 100HV, as measured by Vickers Hardness testing method, and more suitably exhibits a hardness of at least 250HV.

Generally the sintered hydroxyapatite of use in the invention will be in a very fine particulate form. The sintered hydroxyapatite particles will typically have an average particle size of less than about 20 microns and greater than about 0.5 microns. Suitably sintered hydroxyapatite particles of use in the invention will have an average particle size in the range from about 1 micron to about 20 microns, more suitably in the range from about 2 to about 15 microns. Nanosized particles (also known as nanoparticles) of sintered hydroxyapatite having an average particle size of less than 100 nm, typically between 1 and 100 nm, are generally not favoured for use in the present invention. Whilst not being bound by any theory, it is believed that cleaning power may be adversely affected, when the particle size of the sintered hydroxyapatite is very small. Accordingly nanoparticles of sintered hydroxyapatite of less than about 100 nm, for example as described in CN1429538A, and sintered calcium phosphate particles having an average particle diameter in the range of 20-100 nm, as described in US 2012/0064343 (Tamaki et al), are not recommended for use in the present invention.

AU518908B2 describes a translucent, isotropic, polycrystalline, sintered ceramic comprising substantially pure hydroxylapatite having an average crystallite size in the range 0.2 to 3 microns, for use in dental restorative compositions as a dense filler material having a coefficient of expansion virtually identical to that of natural tooth enamel. However there is no disclosure of use of sintered hydroxyapatite in a dentifrice.

The shape of the sintered hydroxyapatite particles may be classified as either angular or spherical or a combination of both. By the term “spherical” herein is meant to include any particle wherein the whole particle is mostly rounded or elliptical in shape. Thus the term “spherical” is meant to include particles that essentially do not have sharp or jagged edges, and are in the shape of spheres, spheroids, ellipsoids, ovoids and the like. By the term “angular” is meant to include any particle that is not spherical, including polyhedral shapes. The angular particles may have some rounded edges, some or all sharp edges, some or all jagged edges, or a combination thereof.

Sintered hydroxyapatite suitable for use in the invention is available commercially from Plasma Biotal Ltd and is sold under the trade name Captal S. A commercially available grade includes Captal S OD.

Suitably the sintered hydroxyapatite is present in an amount from about 0.05 to about 4%, for example from about 0.1 to about 3% or from about 0.2 to about 2.5% or from about 0.75% to about 2.0% by weight of the total dentifrice composition.

A dentifrice composition according to the invention may further comprise a supplementary abrasive agent, provided that such agent(s) do(es) not significantly adversely impact on dentine abrasion. Suitable examples of supplementary abrasive agents for use in the present invention include silica, alumina, hydrated alumina, calcined alumina, calcium carbonate, anhydrous dicalcium phosphate, dicalcium phosphate dihydrate, water-insoluble sodium metaphosphate, zirconia, perlite, diamond, rice hull silica, silica gels, aluminium silicates, pyrophosphates, pumice, polymer particles, calcium phosphate based minerals (e.g. tricalcium phosphate (TCP), hydrated HA and mixed phase (HA:TCP) calcium phosphate mineral) and /or any other whitening agent and mixtures thereof. A supplementary abrasive agent may be used generally in an amount ranging from about 0.1% w/w to about 50% w/w or about 0.1% w/w to about 20% w/w by weight of the total dentifrice composition.

In one embodiment the sintered hydroxyapatite is the sole abrasive agent in the dentifrice composition.

Traditionally, effective whitening has been associated with high dentine abrasivity (RDA) values. The RDA of a particular substance or formulation can be readily determined by one skilled in the art. See “A Laboratory Method for Assessment of Dentifrice Abrasivity” John J. Hefferen, J. Dent. Res, Vol. 55, No. 4, 563-573.

The technology of the present invention allows compositions to maximise cleaning and provide effective whitening with a significantly lower RDA value than traditional whitening toothpastes.

Advantageously a dentifrice composition according to the present invention will comprise a low RDA value ranging from about 10 to about 90, suitably in the range of from about 20 to about 70.

The cleaning ability of dentifrice compositions may be demonstrated using the Pellicle Cleaning Ratio (PCR) test—a laboratory method accepted as useful in the characterization of stain cleaning (whitening) actions of abrasive-containing dentifrices. The PCR value is calculated relative to a standard material (Ca₂P₂O₇, Odontex Inc.) which is given the empirical value of 100.

Advantageously a dentifrice composition according to the present invention will comprise a PCR value ranging from about 50 to about 130, suitably in the range from about 60 to about 120.

Surprisingly a sintered hydroxyapatite for use in the invention has been shown to produce significantly superior cleaning when compared to a standard non-sintered hydroxyapatite, as demonstrated herein in a PCR test. Further the sintered hydroxyapatite has also been shown to deliver cleaning that is equivalent or superior to that observed with a commercial whitening toothpaste. Whilst the commercial whitening toothpaste, comprising silica as the cleaning agent, also demonstrated good cleaning performance, this was at the expense of significantly higher dentine abrasion.

A dentifrice composition according to the present invention shows good cleaning and whitening of the tooth surface, with minimimal dentine abrasivity. Such compositions also have acceptable enamel abrasivity. The high cleaning/low abrasivity properties of a dentifrice composition according to the invention may also be reflected in the Cleaning Efficiency Index value for the dentifrice composition. The Cleaning Efficiency Index value can be readily determined by one skilled in the art. See Schemehorn B R, Ball T L, Henry G M, Stookey G K. “Comparing dentifrice abrasive systems with regard to abrasion and cleaning ” J. Dent Res 1992; 71:559.

Advantageously a dentifrice composition according to the present invention will comprise a CEI value ranging from about 1.25 to about 2.6, suitably in the range from about 1.4 to about 2.4.

A dentifrice composition of the present invention can therefore provide, after brushing, cleaner, whiter and smoother tooth surfaces having less plaque, tartar and/or stain thereby resulting in an overall improvement in oral health.

A dentifrice composition of the present invention may further comprise a water-soluble condensed phosphate salt, such as an alkali metal pyrophosphate, tripolyphosphate or higher polyphosphate salt, in particular a water soluble alkali metal tripolyphosphate salt. Suitably the sodium form of this salt is preferred, although the potassium or mixed sodium and potassium salts could be used as a preferred embodiment as well. All physical forms can be used, e.g. a hydrate or the dehydrated form.

Most suitably the water soluble alkali metal tripolyphosphate salt is sodium tripolyphosphate.

Suitably the water soluble condensed phosphate salt (such as an alkali metal tripolyphosphate salt) is present in an amount from about 1.0% to about 20.0%, for example from about 2.0% to about 15.0% or about 5.0% to about 10.0% by weight of the total composition.

A dentifrice composition of the present invention may comprise one or more active agents conventionally used in dentifrice compositions, for example, a fluoride source, a desensitising agent, an anti-bacterial agent, an anti-plaque agent, an anti-calculus agent, an oral malodour agent, an anti-inflammatory agent, an anti-oxidant, an anti-fungal agent, wound healing agent or a mixture of at least two thereof. Such agents may be included at levels to provide the desired therapeutic effect.

Examples of desensitising agents include a tubule blocking agent or a nerve desensitising agent and mixtures thereof, for example as described in WO02/15809 (Block). Examples of nerve desensitising agents include a strontium salt such as strontium chloride, strontium acetate or strontium nitrate or a potassium salt such as potassium citrate, potassium chloride, potassium bicarbonate, potassium gluconate and especially potassium nitrate.

A desensitising agent such as a potassium salt is generally present between from about 2% to about 8% by weight of the total composition, for example about 5% by weight of potassium nitrate may be used.

In one embodiment the desensitizing agent comprises a tubule blocking agent such as a bioactive glass. Suitably the bioactive glass consists of about 45% by weight silicon dioxide, about 24.5% by weight sodium oxide, about 6% by weight phosphorus oxide, and about 24.5% by weight calcium oxide. One such bioactive glass is available commercially under the trade name, NovaMin®, also known as 45S5 Bioglass®.

Suitably the bioactive glass is present in an amount ranging from about 1 to about 20% by weight of the dentifrice composition, such as from about 1 to about 15%, or such as from about 1 to about 10%, or such as from about 2 to about 8% by weight of the dentifrice composition.

In another embodiment the desensitizing agent comprises a tubule blocking agent such as an arginine calcium carbonate salt. Suitably the arginine salt is present in an amount ranging from about 0.5% w/w to 30% w/w of the dentifrice composition, such as from about 1 to 10% w/w, or such as from about 1 to about 10% w/w, or such as from about 2 to about 8% w/w of the dentifrice composition.

In another embodiment the desensitizing agent comprises a tubule blocking agent such as an acid oxalate, alkali metal or alkaline earth metal oxalate. Suitable oxalates for use in a dentifrice composition of the invention may include soluble and sparingly soluble oxalates such as sodium, potassium, lithium, calcium, magnesium, barium and strontium. In one aspect the oxalate is selected from sodium oxalate, potassium oxalate, calcium oxalate and mixtures thereof. Suitably the oxalate salt is present in an amount ranging from 0.0025% to 10% by weight of the dentifrice composition.

In a further embodiment the desensitizing agent includes a tubule blocking agent, such as a silica, colloidal silica, nano zinc oxide, sub-micron alumina and sub micron polymer beads, in a fine particulate form comprising an average particle size in the range from about 1 nm to about 5 microns.

Suitable sources of fluoride ions for use in the compositions of the present invention include an alkali metal fluoride such as sodium fluoride, an alkali metal monofluorophosphate such a sodium monofluorophosphate, stannous fluoride, or an amine fluoride in an amount to provide from about 25 to about 3500 pm of fluoride ions, preferably from about 100 to about 1500 ppm. A typical fluoride source is sodium fluoride, for example the composition may contain from about 0.1 to 0.5% by weight of sodium fluoride, eg 0.204% by weight (equating to 923 ppm of fluoride ions), 0.2542% by weight (equating to 1150 ppm of fluoride ions) or 0.315% by weight (equating to 1426 ppm of fluoride ions).

Such fluoride ions help promote the remineralisation of teeth and can increase the acid resistance of dental hard tissues for combating caries, dental erosion (ie. acid wear) and/or tooth wear.

Compositions of the present invention will contain additional formulating agents such as, surfactants, humectants, thickening agents such as non-abrasive (thickening) silicas, flavouring agents, sweetening agents, opacifying or colouring agents, preservatives and water, selected from those conventionally used in the oral hygiene composition art for such purposes.

Suitable surfactants for use in the present invention include anionic surfactants such as a sodium C₁₀₋₁₈ alkyl sulphate, e.g. sodium lauryl sulphate, and a taurate surfactant. Sodium lauryl sulphate is generally considered to be anionic and strongly charged and is useful if high levels of foaming are desired when brushing teeth. Taurate surfactants useful in the present invention are salts of fatty acid amides of N-methyl taurine. They conform generally to the structural formula:

RC(O)N(CH₃)CH₂CH₂SO₃M

Where RC(O)- represents a fatty acid radical and M represents sodium, potassium, ammonium or triethanolamine. Fatty acids having carbon chain lengths of from 10 to 20, including those derived from coconut, palm and tall oil may be used. In one embodiment the fatty acid is derived from coconut. In one embodiment, sodium salts are used. In one embodiment the taurate is sodium methyl cocoyl taurate. This taurate surfactant is sold under the trademark Adinol CT by Croda, such as Adinol CT95.

In addition to anionic surfactants, zwitterionic, amphoteric, cationic and non- or low-ionic surfactants may be used to aid foaming characteristics. When anionic and amphoteric surfactants are used together an optimised foaming system is achieved that will provide both improved mouth feel and good cleaning Examples of amphoteric surfactants include long chain alkyl (eg. C₁₀-C₁₈ alkyl) betaines, such as the product marketed under the tradename ‘Empigen BB’ by Albright & Wilson and long chain alkyl amidoalkyl betaines such as cocamidopropylbetaine.

A particularly preferred example of an anionic/amphoteric surfactant combination for use in the present invention is a C₁₀₋₁₈ alkyl sulphate/C₁₀-C₁₈ alkyl betaine, such as sodium lauryl sulphate/cocamidopropylbetaine.

In an alternative embodiment an anionic/amphoteric surfactant combination for use in the invention is a taurate/C₁₀-C₁₈ alkyl betaine, such as sodium methyl cocoyl taurate/cocamidopropylbetaine.

Suitably, the surfactant is present in the range from about 0.1 to about 15%, for example from about 0.5 to about 10% or from about 1.0 to about 5% or from about 0.5 to about 2% by weight of the total composition.

Suitable humectants for use in compositions of the invention include glycerin, xylitol, sorbitol, propylene glycol or polyethylene glycol, or mixtures of at least two thereof for example a mixture comprising glycerin, sorbitol and polyethylene glycol; which humectant may be present in the range from about 10 to about 80%, for example from about 20 to about 70% or from about 30 to about 60% by weight of the total composition.

Suitable thickening agents for use in compositions of the invention include carboxyvinyl polymers, carrageenan, alginate, hydroxyethyl cellulose, and water soluble salts of cellulose ethers such as sodium carboxymethylcellulose. Natural gums such as gum karaya, xanthan gum, gum Arabic and gum tragacanth can also be used. Colloidal magnesium aluminium silicate or finely divided silica can be used as part of the thickeneing agent. Thickening agents can be used from about 0.1% to about 15% by weight of the total composition.

It will be understood that compositions of the present invention may also be used outside the oral cavity, for the cleaning of dentures and the like.

The dentifrice composition of the present invention are typically formulated in the form of a paste and/or a gel that is suitable for containing in and dispensing from a laminate tube or a pump as conventionally used in the art. Additional examples may include bag-in-can or bag-on-valve delivery systems that utilise a foaming agent such as pentane or iso-pentane.

A typical process for making the composition of this invention involves admixing the ingredients, suitably under a vacuum, until a homogeneous mixture is obtained, and adjusting the pH if necessary.

Each of the aspects of the invention are independent unless stated otherwise. Nevertheless the skilled person will understand that all the permutations of the aspects described are within the scope of the invention. Thus it is to be understood that the present invention covers all combinations of suitable, convenient and exemplified groups described herein.

The invention is further illustrated by the following Examples.

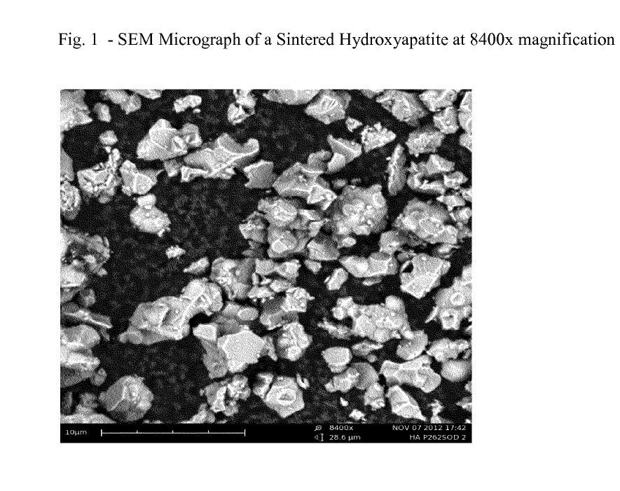

FIG. 1 is an SEM micrograph of a sintered hydroxyapatite at 8400× magnification. The sample comprises dry sintered hydroxyapatite powder.

### EXAMPLES

Data from the following formulations were obtained.

| | Dentifrice Composition | | | | | | | | |
|---|---|---|---|---|---|---|---|---|---|
| | Ex 1 | Ex 2 | Ex 3 | Ex 4 | Ex 5 | Ex 6 | Ex 7 | Ex 8 | Ex 9 |
| Material | % w/w | % w/w | % w/w | % w/w | % w/w | % w/w | % w/w | % w/w | % w/w |
| Sorbitol, Liquid | 28.5 | 28.5 | 28.5 | 28.5 | 28.5 | 28.5 | 28.5 | 28.5 | 28.5 |
| (Non- | | | | | | | | | |
| Crystallising) | | | | | | | | | |
| Glycerol (Ph. | 7.6 | 7.6 | 7.6 | 7.6 | 7.6 | 7.6 | 7.6 | 7.6 | 7.6 |
| Eur) | | | | | | | | | |
| Polyethylene | 3 | 3 | 3 | 3 | 3 | 3 | 3 | 3 | 3 |
| Glycol 300 | | | | | | | | | |
| Sintered HA | 0.25 | 0.5 | 0.75 | 1 | 1.5 | 2 | 1.5 | 0 | 0 |
| Std HA | 0 | 0 | 0 | 0 | 0 | 0 | 0 | 1.5 | 1.5 |
| Hydrated silica | 16.25 | 16 | 15.75 | 15.5 | 15 | 14.5 | 14.5 | 15 | 15 |
| Sodium | 0 | 0 | 0 | 0 | 0 | 0 | 1 | 0 | 0 |
| Tripolyphosphate | | | | | | | | | |
| Cocamidopropyl | 0.6 | 0.6 | 0.6 | 0.6 | 0.6 | 0.6 | 0.6 | 0.6 | 0 |
| betaine | | | | | | | | | |
| Adinol CT95 | 0.6 | 0.6 | 0.6 | 0.6 | 0.6 | 0.6 | 0.6 | 0.6 | 0 |
| Sodium | 0 | 0 | 0 | 0 | 0 | 0 | 0 | 0 | 1.2 |
| laurylsulphate | | | | | | | | | |
| Xanthan Gum | 0.8 | 0.8 | 0.8 | 0.8 | 0.8 | 0.8 | 0.8 | 0.8 | 0.8 |
| Carrageenan | 0.4 | 0.4 | 0.4 | 0.4 | 0.4 | 0.4 | 0.4 | 0.4 | 0.4 |
| Saccharin | 0.3 | 0.3 | 0.3 | 0.3 | 0.3 | 0.3 | 0.3 | 0.3 | 0.3 |
| Sodium | | | | | | | | | |
| Sodium Fluoride | 0.3152 | 0.3152 | 0.3152 | 0.3152 | 0.3152 | 0.3152 | 0.3152 | 0.3152 | 0.3152 |
| Titanium | 0.6 | 0.6 | 0.6 | 0.6 | 0.6 | 0.6 | 0.6 | 0.6 | 0.6 |
| Dioxide | | | | | | | | | |
| Flavour | 1 | 1 | 1 | 1 | 1 | 1 | 1 | 1 | 1 |
| Sodium | 0.225 | 0.225 | 0.225 | 0.225 | 0.225 | 0.225 | 0.225 | 0.225 | 0.225 |
| hydroxide | | | | | | | | | |
| Purified Water | ad 100 | ad 100 | ad 100 | ad 100 | ad 100 | ad 100 | ad 100 | ad 100 | ad 100 |

Examples 1 to 7 fall within the scope of the present invention comprising a sintered hydroxyapatite. Examples 8 and 9 are comparative examples comprising a standard non-sintered hydroxyapatite. In addition to the above experimental formulations which correspond to the first nine examples shown in the graphs below, Sensodyne Gentle Whitening was also tested.

### PCR Methodology

### Specimen Preparation

Bovine, permanent, central incisors were cut to obtain labial enamel specimens approximately 10×10 mm. The enamel specimens were then embedded in an autopolymerizing methacrylate resin so that only the enamel surfaces were exposed. The enamel surfaces were then smoothed and polished on a lapidary wheel and lightly etched to expedite stain accumulation and adherence. They were placed on a rotating rod (in 37° C. incubator) alternately exposing them to air and to a solution consisting of trypticase soy broth, tea, coffee, mucin, FeCl3, and Micrococcus luteus BA13. The staining broth was changed and specimens rinsed daily for seven days. After seven days, a darkly stained pellicle film was apparent on the enamel surfaces. Specimens were then rinsed, allowed to air dry, and refrigerated until use. All products were tested using specimens prepared at the same time.

### Scoring and Set-Up

The amount of in vitro stain was graded photometrically using only the L value of the L*a*b* scale using a spectrophotometer (Minolta CM2600d.) The area of the specimens scored was a ¼-inch diameter circle in the center of the 10×10 mm enamel. Specimens with scores between 30 and 42 (30 being more darkly stained) were used. On the basis of these scores, the specimens were divided into groups of 16 specimens each, with each group having the same average baseline score.

### Test Procedure

The specimens were then mounted on a mechanical V-8 cross-brushing machine equipped with soft nylon-filament (Oral-B 40) toothbrushes. Brush force on the enamel surface was adjusted to 150 g. The dentifrices were used as slurries prepared by mixing 25 grams of dentifrice with 40 ml of deionized water. The ADA abrasion reference material (Ca₂P₂O₇) was prepared by mixing 10 g in 50 ml of a 0.5% CMC solution. The specimens were brushed for 800 strokes (4½ minutes). To minimize mechanical variables, two specimens per group were brushed on each of the eight brushing heads. Fresh slurries were made after being used to brush four specimens. Following brushing, specimens were rinsed, blotted dry, and scored again for stain as previously described.

### Calculations

The difference between the pre- and post-brushing stain scores was determined and the mean and standard error calculated for the reference group. The cleaning ratio for the reference material group was assigned a value of 100. The mean decrement of the reference group was divided into 100 to obtain a constant value to multiple times each individual test decrement within the study. The individual cleaning ratio of each specimen was then calculated (decrement X constant). The mean and SEM for each group (N=16) was then calculated using the individual cleaning ratios. The larger the value of the cleaning ratio, the greater the amount of stained pellicle removed in this test.

These PCR results (see Graph 1) demonstrate that sintered hydroxyapatite produced superior cleaning compared to standard hydroxyapatite and when used at 0.75% w/w and above has the potential to deliver excellent cleaning that is parity or better than a typical commercial whitening toothpaste. Whilst the commercial toothpaste formulation, comprising silica, also has good cleaning performance this is at the expense of higher levels of abrasivity as determined in the RDA methodology described below.

### RDA Methodology

### Specimen Preparation

The procedure used in this study was the Hefferren abrasivity test recommended by the ADA and ISO 11609 for determination of dentifrice relative abrasiveness in dentin. Eight (8) human dentin specimens were subjected to neutron bombardments resulting in the formation of radioactive phosphorus (32P) within the specimens under the controlled conditions outlined by the ADA. The specimens were then mounted in methyl methacrylate so they would fit in a V-8 cross-brushing machine. The specimens were preconditioned by brushing for 5000-strokes, (soft Oral B-40; 150g brush tension) using a slurry consisting of 10 g ADA reference material in 50 ml of a 0.5% CMC glycerin solution.

### Procedure

Following the precondition run, the test was performed (150 g and 1500 strokes) using in a “sandwich design.” Before and after being brushed with the test product (25 g/40 ml water) each tooth set was brushed with the ADA reference material (10 g of Ca₂P₂O₇/50 ml 0.5% CMC). The procedure was repeated additional times so that each product was assayed on each tooth set. The treatment design was a modified Latin Square design so that no treatment followed another treatment consistently.

### Calculations

One ml samples were taken, weighed (˜1 g), and added to 4.5 ml of “Ultima Gold” scintillation cocktail. The samples were mixed well and immediately put on the scintillation counter for radiation count. Following counting, the net counts per minute (CPM) values were divided by the weight of the sample to calculate the net CPM/gram per slurry. The net CPM/g of the pre and post ADA reference material for each of the test slurries was then calculated and averaged to use in the calculation of RDA (relative dentin abrasion) for the test material. The ADA reference material was assigned a value of 100 and its ratio to the test material was calculated.

The RDA results (see Graph 2) illustrate that the sintered and standard hydroxyapatite produce extremely low levels of dentine abrasivity compared to the commercial product. The values obtained in the above PCR and RDA experiments were then used to obtain the following Cleaning Efficiency Index values (CEI=(PCR-50+RDA)/RDA). Ref: Schemehorn B R, Ball T L, Henry G M, Stookey G K. Comparing dentifrice abrasive systems with regard to abrasion and cleaning. J Dent Res 1992;71: 559.

The Cleaning Efficiency Index data (See Graph 3) demonstrate that compositions of the present invention containing a sintered hydroxyapatite agent provide highly effective cleaning at low RDA levels; certainly more effectively than either formulations containing standard hydroxyapatite or a typical commercial formulation containing silica abrasive and a condensed polyphosphate.

1. A dentifrice composition comprising a sintered hydroxyapatite abrasive agent.

2. The composition according to claim 1 wherein the average particle size of the sintered hydroxyapatite is in the range from about 1 to about 20 microns.

3. The composition according to claim 1 wherein the sintered hydroxyapatite is present in an amount from 0.05 to 4% by weight of the total dentifrice composition.

4. The composition according to claim 1 comprising a supplementary abrasive agent.

5. The composition according to claim 4 wherein the supplementary abrasive agent is selected from silica, alumina, hydrated alumina, calcined alumina, calcium carbonate, anhydrous dicalcium phosphate, dicalcium phosphate dehydrate, water-insoluble sodium metaphosphate, zirconia, perlite, diamond, rice hull silica, silica gels, aluminium silicates, pyrophosphates, pumice, calcium phosphate based mineral (e.g. tricalcium phosphate (TCP), hydrated HA and mixed phase (HA:TCP) calcium phosphate mineral) and mixtures thereof.

6. The composition according to claim 1 wherein the sintered hydroxyapatite is the sole abrasive agent in the dentifrice composition.

7. The composition according to claim 1 comprising a water-soluble condensed phosphate.

8. The composition according to claim 7 wherein the condensed phosphate is a water-soluble alkali metal tripolyphospate salt.

9. The composition according to claim 1 comprising a desensitizing agent.

10. The composition according to claim 1 claims comprising a source of fluoride ions.

11. (canceled)

12. The process for preparing a dentifrice composition according to claim 1 involving admixing the ingredients, suitably under a vacuum, until a homogenous mixture is obtained, and adjusting the pH if necessary.
